(19) United States
(12) Patent Application Publication
(10) Pub. No.: US 2012/0029162 A1
(43) Pub. Date: Feb. 2, 2012

(54) CARBON NANOTUBES COMPRISING HYDROXY GROUPS, METHOD FOR THE PRODUCTION THEREOF AND POLYURETHANE POLYMERS COMPRISING SAID CARBON NANOTUBES

(75) Inventors: Stephanie Vogel, Langenfeld (DE); Hartmut Nefzger, Pulheim (DE); Jörg Hofmann, Krefeld (DE)

(73) Assignee: Bayer Materialscience AG, Leverkusen (DE)

(21) Appl. No.: 13/262,935

(22) PCT Filed: Mar. 30, 2010
(86) PCT No.: PCT/EP10/02002

§ 371 Date: Oct. 18, 2011

(30) Foreign Application Priority Data
Apr. 9, 2009 (EP) 09005186.3

(51) Int. Cl. C08G 18/66; C08G 18/08; C07C 69/76; B82B 1/00; B82Y 40/00; B82B 3/00; B82Y 30/00

(52) U.S. Cl. 528/76; 560/8; 528/80; 977/748; 977/847

(57) ABSTRACT

The invention relates to carbon nanotubes comprising hydroxy groups, wherein the surface thereof comprises hydroxy alkyl ester groups covalently bound thereon, chosen from the group according to the general formula (1) and/or the general formula (2), wherein (CNT) stands for the surface of the carbon nanotube and R1 and R2 are independently from each other hydrocarbon, an alkyl radical or an aryl radical. The invention further relates to a method for the production thereof by means of reaction of carbon acid groups of the carbon nanotubes with an epoxy, furthermore a polyurethane polymer, wherein said carbon nanotubes are covalently bound, a method for producing a polymer of said kind and use of the carbon nanotubes for producing polymers.

The present invention relates to carbon nanotubes comprising hydroxyl groups, the surface thereof comprising hydroxyalkyl ester groups bonded covalently thereto. It further relates to a process for preparing carbon nanotubes comprising such hydroxyl groups, to a process for preparing polyurethane polymers comprising carbon nanotubes and to a polyurethane polymer comprising carbon nanotubes, wherein at least some of the carbon nanotubes are bonded covalently to the polyurethane polymer.

Carbon nanotubes (CNTs) are known for their unusual properties. For example, the strength thereof is about 100 times that of steel, the thermal conductivity thereof is about twice as great as that of diamond, the thermal stability thereof ranges up to 2800° C. in vacuum and the electrical conductivity thereof may be several times the conductivity of copper. These structure-related characteristics are, however, obtainable at the molecular level only when it is possible to distribute carbon nanotubes homogeneously and to establish maximum contact between tubes and the medium, i.e. to make them compatible with the medium and hence dispersible in a stable manner.

A chemical functionalization of carbon nanotubes or carbon fibers can improve the dispensability thereof, among other properties. A review article by N. Tsubokawa (Polymer Journal 2005, 37, 637-655) lists a multitude of options for such a functionalization. In addition to complex chemical reactions, for example ligand exchange reactions on 1,1′-dicarboxyferrocene, living free-radical polymerization with polystyrene and reaction with azides, the oxidation of the carbon nanotubes with HNO₃, which is known in this field, and corresponding modifications based thereon were also mentioned. Tsubokawa reports three options for the chemistry following the oxidative introduction of carboxyl groups.

The first variant for this purpose is the activation of the acid groups by thionyl chloride, followed by a further reaction with a nucleophile. A disadvantage thereof is the SO₂ and HCl released by the use of thionyl chloride. The second variant is the reaction of the carboxyl groups of the carbon nanotubes with coupling reagents, such as DCC known from peptide chemistry, with subsequent reaction with a nucleophile. However, this method requires the use of an expensive coupling reagent and produces, according to the coupling reagent used, a sparingly soluble urea as a byproduct.

Finally, a third variant described in this review article is based on carbon fibers obtained from the gas phase (vapor grown carbon fibers, VGCF). In this case, an anionic polymerization of epoxides and acid anhydrides fakes place as a result of alternating ring openings. This has already been described separately by the same author in Polymer Journal 2004, 36, 316-322. The synthesis sequence is initiated by the deprotonation of the carboxyl group by KOH. As a result, the polymerization has to be performed in the presence of crown ethers, which makes this chemistry very expensive and entails waste problems. In the specific example, the carboxylate group of the carbon fibers is reacted with styrene oxide and phthalic anhydride. It can be inferred from a table that the reaction of the carboxylate group with styrene oxide alone does not lead to any reaction.

The functionalization of carboxyl groups in the polyester polyol synthesis is disclosed in DE 36 13 875 A1. To prepare polyester polyols with an acid number of less than 1, a hydroxyl number of about 20 to about 400 and a functionality of appropriately 2 to 3, polycarboxylic acids and/or anhydrides thereof and polyhydric alcohols are condensed. This is advantageously done in the absence of customary esterification catalysts at temperatures of 150° C. to 250° C. and optionally under reduced pressure. Polycondensation is effective up to an acid number of 20 to 5 and the resulting polycondensates are then alkoxylated with 1 to 5 mol per carboxyl group of alkylene oxide, for example 1,2-propylene oxide and/or preferably ethylene oxide, in the presence of a tertiary amine. The tertiary amine is selected from the group of N-methylimidazole, diazabicyclo[2.2.2]octane, diazabicyclo[5.4.0]undec-7-ene and pentamethylenediethylenetriamine. The catalyst is appropriately used in an amount of 0.001 to 1.0% by weight, based on the weight of polycondensate. Alkoxylation is advantageously effected at temperatures of 100° C. to 170° C. and under a pressure of 1 to 10 bar.

There has to date been no description of functionalized carbon nanotubes in which, based on carboxyl groups present on the surface, hydroxyalkyl ester groups with a distance of 2 carbon atoms between the ester function and the free OH group are present. Such carbon nanotubes bearing hydroxyl groups would be useful for functionalizations in which attachment via an OH group is required due to the specific chemical circumstances.

The invention therefore proposes a carbon nanotube comprising hydroxyl groups, wherein the surface thereof comprises hydroxyalkyl ester groups which are bonded covalently thereto and are selected from the group as claimed in the general formula (1) and/or the general formula (2), where (CNT) represents the surface of the carbon nanotube and R1 and R2 are each independently hydrogen, an alkyl radical or an aryl radical:

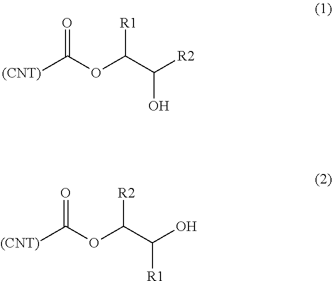

The structures according to the isomeric general formulae (1) and (2) can be derived from the reaction of carboxyl or carboxylate groups present on the surface of the carbon nanotubes with epoxides bearing the R1 and R2 radicals. In this context, opening of the epoxide ring affords a carboxylic ester with an OH group at a distance of 2 carbon atoms from the ester group. According to how the epoxide ring opens, the ester group is attached to the R1-bearing carbon atom according to formula (1) or to the R2-bearing carbon atom according to formula (2). When one of the R1 and R2 radicals is hydrogen and the other of the two radicals is not, primary or secondary alcohols can be obtained.

The term “alkyl” generally encompasses, in the context of the entire invention, substituents from the group of N-alkyl, branched alkyl and/or cycloalkyl. The term “aryl” generally encompasses, in the context of the overall invention, substituents from the group of monocyclic carbo- or heteroaryl substituents and/or polycyclic carbo- or heteroaryl substituents.

The free OH group obtained can then be used for further reactions in order to further functionalize the surface of the carbon nanotube. The content of these free OH groups, expressed in mmol of OH groups per gram of carbon nanotubes, may, for example, be ≧0.1 mmol/g to ≦5 mmol/g, preferably ≧0.5 mmol/g to ≦1 mmol/g.

In one embodiment of the inventive carbon nanotube comprising hydroxyl groups, R1 and R2 are each independently hydrogen, methyl, ethyl, n-propyl, isopropyl, n-butyl, sec-butyl, isobutyl, tert-butyl and/or phenyl, or together are —(CH₂)₄—. When R1 and R2 together are —(CH₂)₄—, the structure of such inventive carbon nanotubes would be derived from the reaction of carboxyl or carboxylate groups present on the surface of the carbon nanotubes with cyclohexane oxide. It is preferred, however, that R1 and R2 are each hydrogen, or else that R1 is hydrogen and R2 is methyl. In that case, the structure of such inventive carbon nanotubes can be attributed to the reaction of carboxyl or carboxylate groups present on the surface of the carbon nanotubes with ethylene oxide or propylene oxide.

In a further embodiment of the inventive carbon nanotube comprising hydroxyl groups, wherein the carbon nanotube is selected from the group comprising single-wall carbon nanotubes, multiwall carbon nanotubes, and multiwall carbon nanotubes of the cylinder type, of the scroll type, of the multiscroll type and/or with onion-like structure. It is favorable when the carbon nanotubes have a ratio of length to external diameter of ≧5, preferably ≧100.

In contrast to the already mentioned known carbon nanotubes of the scroll type with only one continuous or interrupted graphene layer, there also exist carbon nanotube structures which consist of several graphene layers which have been combined to give a stack and are present in rolled-up form. Reference is made in this context to the multiscroll type. These carbon nanotubes are described in DE 10 2007 044031 A1, to which full reference is made. This structure with respect to the carbon nanotubes of the simple scroll type is comparable to the structure of multiwall cylindrical carbon nanotubes (cylindrical MWNTs) relative to the structure of the single-wall cylindrical carbon nanotubes (cylindrical SWNTs).

It is favorable when the carbon nanotube comprising inventive hydroxyl groups has a diameter of ≧3 nm to ≦100 nm. The diameter is based here on the mean diameter of the carbon nanotubes. It may also be within a range from ≧5 nm to ≦80 nm and advantageously from ≧6 nm to ≦60 nm. The length of the carbon nanotubes is basically unlimited. It may, however, for example, be within a range from ≧1 μm to ≦100 μm and advantageously from ≧10 μm to ≦30 μm.

The present invention further provides a process for preparing inventive carbon nanotubes comprising hydroxyl groups, comprising the steps of:
(a) providing carbon nanotubes whose surface comprises carboxyl groups bonded covalently thereto; and
(b) reacting the carbon nanotubes from step (a) with an epoxide

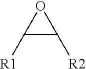

where R1 and R2 are independently hydrogen, an alkyl radical or an aryl radical.

This synthesis route is at least one step shorter compared to the esterification via carbonyl chlorides on the surface of the carbon nanotubes. It is also particularly advantageous that this synthesis can be performed under comparatively mild reaction conditions. In general, distinctly higher temperatures are needed for a direct esterification of carboxyl groups on the surface of a carbon nanotube, as a result of which the reagglomeration of the nanotubes can take place, or else the individualization of the nanotubes does not occur due to the chemical functionalization. Relatively large nanotube agglomerates with diameters in the range from 100 μm to 200 μm then have to be removed in a complex filtration process. Otherwise, preferential fracture sites would be obtained after a further processing, for example in a polymer molding. Compared to the acid chloride route, a further advantage is that the process according to the invention is an addition reaction which has to proceed only with small amounts of a catalyst. In the esterification via an acid chloride, in contrast, stoichiometric amounts of hydrogen chloride would be released, which would ultimately have to be removed.

The inventive carbon nanotube comprising hydroxyl groups can be described such that it is obtainable from this process according to the invention.

The carbon nanotubes which comprise carboxyl groups bonded covalently to the surface and used as starting material can be obtained from unfunctionalized carbon nanotubes by means of oxidative processes such as the HNO₃ process. The content of carboxyl groups at the surface can be determined by conductometric titration and reported in mmol of carboxyl groups per gram of carbon nanotubes. The content may be, for example, ≧0.1 mmol/g to ≦5 mmol/g, preferably ≧0.5 mmol to ≦1 mmol/g.

The provision of carbon nanotubes in step (a) is effected advantageously as a dispersion in a solvent. This solvent should accordingly be selected such that it does not enter into any unwanted side reactions with the epoxide used. Suitable solvents include aromatic hydrocarbons such as toluene or xylene, cycloalkanes such as cyclohexane, alkanes such as hexane, and ethers, especially cyclic ethers such as tetrahydrofuran or dioxane. The concentration of the carbon nanotubes in a dispersion may, for example, be within a range from ≧0.1% by weight to ≦10% by weight, preferably from ≧1% by weight to ≦5% by weight. The dispersion can be obtained by using a stirrer with a rotor/stator system at high speeds, for example between ≧20 000 rpm and ≦25 000 rpm. In addition, ultrasound can act on the dispersion.

In order to drive the reaction in step (b) in the desired direction, the epoxide can be used in a large excess relative to the carboxyl groups on the surface of the carbon nanotubes. If the epoxide is gaseous under the reaction conditions, the reaction pressure (absolute) may, for example, be from ≧2 bar to ≦5 bar.

In one embodiment of this process, the reaction of the carbon nanotubes in step (b) is performed in the presence of a tertiary amine as a catalyst. Preference is given here to tertiary diamines. Examples thereof are diazabicycloalkanes such as 1,4-diazabicyclo[2.2.2]octane, diazabicycloalkenes such as 1,8-diazabicyclo[5.4.0]undec-7-ene, triamines such as pentamethylenediethylenetriamine, and bis(2-dialkylaminoalkyl)ethers such as bis(2-dimethylaminoethyl)ether. Especially the bis(2-dimethylaminoethyl)ether catalyst leads to the effect that only one molecule of the epoxide reacts with the carboxyl group, such that there is no formation of polyether chains.

In a further embodiment of this process, R1 and R2 in the epoxide in step b) are each independently hydrogen, methyl, ethyl, n-propyl, isopropyl, n-butyl, sec-butyl, isobutyl, tert-butyl and/or phenyl, or together are —(CH₂)₄—. The epoxide in step (b) is preferably a terminal alkylene oxide. In that case, at least one of the R1 and R2 radicals is hydrogen. Examples of such alkylene oxides are ethylene oxide, propylene oxide and n-butylene oxide. Preference is given here to ethylene oxide and propylene oxide.

In a further embodiment of this process, the carbon nanotubes in step (a) are selected from the group comprising single-wall carbon nanotubes, multiwall carbon nanotubes, and multiwall carbon nanotubes of the cylinder type, of the scroll type, of the multiscroll type and/or with onion-like structure. Advantageously, the carbon nanotubes in step (a) have a diameter of ≧3 nm to ≦100 nm. Further details and preferred lengths and diameters have already been described above in connection with the inventive carbon nanotubes. Therefore, reference is made to the above for avoidance of repetition.

In a further embodiment of this process, the reaction in step (b) is performed at a temperature of ≧100° C. to ≦150° C. Advantageously, the reaction temperature in this context is between ≧120° C. and ≦130° C. Such comparatively low reaction temperatures enable the functionalization of the carbon nanotubes without the reagglomeration thereof becoming dominant. From a process technology point of view, such temperatures, which are below the temperature generally required for a direct esterification of the carboxyl groups with alcohols, are additionally associated with an energy saving.

The inventive carbon nanotubes comprising hydroxyl groups can advantageously be used additionally in the synthesis of polyurethane polymers and thus incorporated covalently into the polymer matrix. The present invention therefore further provides a process for preparing polyurethane polymers comprising carbon nanotubes, comprising the steps of:
(a) providing a dispersion of carbon nanotubes whose surface comprises carboxyl groups bonded covalently thereto in a polyol;
(b) reacting the dispersion from step (a) with an epoxide

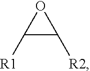

where R1 and R2 are each independently hydrogen, an alkyl radical or an aryl radical; and
(c) reacting the dispersion obtained in step (b) with a polyisocyanate.

Without being bound to a theory, it is assumed that the free hydroxyl groups of the inventive carbon nanotubes obtained after step (b) likewise react at least partly with isocyanate groups during the formation of the polyurethane polymer. The result here is that a polyurethane polymer chain is attached covalently to the surface of the carbon nanotube.

The carbon nanotubes which comprise carboxyl groups bonded covalently to the surface and are used as a starting material can be obtained from unfunctionalized carbon nanotubes by means of oxidative processes such as the HNO₃ process. The content of carboxyl groups at the surface can be determined by conductometric titration and be reported in mmol of carboxyl groups per gram of carbon nanotubes. The content may be, for example, ≧0.1 mmol/g to ≦5 mmol/g, preferably ≧0.5 mmol/g to ≦1 mmol/g.

The carbon nanotubes in step (a) may be selected from the group comprising single-wall carbon nanotubes, multiwall carbon nanotubes, and multiwall carbon nanotubes of the cylinder type, of the scroll type, of the multiscroll type and/or with onion-like structure. The carbon nanotubes in step (a) advantageously have a diameter of ≧3 nm to ≦100 nm. Further details and preferred lengths and diameters have already been described above in connection with the inventive carbon nanotubes. Therefore, reference is made to the above for avoidance of repetition.

The provision of carbon nanotubes in step (a) is effected as a dispersion in a polyol. The concentration of the carbon nanotubes in the dispersion may, for example, be within a range from 0.1% by weight to ≦10% by weight, preferably from ≧1% by weight to ≦5% by weight. The dispersion can be obtained by using a stirrer with a rotor/stator system at high speeds, for example between ≧20 000 rpm and ≦25 000 rpm. In addition, it is possible here for ultrasounds to act on the dispersion.

Suitable polyols are in principle the polyols customary in polyurethane chemistry, for example polyether polyols, polyacrylate polyols, polycarbonate polyols, polycaprolactone polyols, polyurethane polyols and polyester polyols. Such polyols are described in “Ullmanns Enzyklopädie der technischen Chemie”, 4th edition, volume 19, p. 304-305, Verlag Chemie, Weinheim, or in “Polyurethane—Lacke, Kleb- und Dichtstoffe” [Polyurethanes—Coatings, Adhesives and Sealants] by Ulrich Meier-Westhues, Vincentz Network, Hanover, 2007.

In order to drive the reaction in step (b) in the desired direction, the epoxide can be used in a large excess compared to the carboxyl groups on the surface of the carbon nanotubes. If the epoxide is gaseous under the reaction conditions, the reaction pressure (absolute) may, for example, be ≧2 bar to ≦10 bar.

Suitable polyisocyanates are aromatic, araliphatic, aliphatic or cycloaliphatic polyisocyanates having an NCO functionality of ≧2.

Examples of such suitable polyisocyanates are butylene 1,4-diisocyanate, hexamethylene 1,6-diisocyanate (HDI), isophorone diisocyanate (IPDI), 2,2,4- and/or 2,4,4-trimethyl-hexamethylene diisocyanate, the isomeric bis(4,4′-isocyanatocyclohexyl)methanes or mixtures thereof with any isomer content, cyclohexylene 1,4-diisocyanate, phenylene 1,4-diisocyanate, tolylene 2,4- and/or 2,6-diisocyanate (TDI), naphthylene 1,5-diisocyanate, diphenylmethane 2,2′- and/or 2,4′- and/or 4,4′-diisocyanate (MDI), 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) having C₁- to C₈-alkyl groups.

In addition to the aforementioned polyisocyanate, it is also possible to use, in portions or completely, modified diisocyanates with uretdione, isocyanurate, urethane, allophanate, biuret, iminooxadiazinedione and/or oxadiazinetrione structure, and unmodified polyisocyanate with more than 2 NCO groups per molecule, for example 4-isocyanatomethyl-1,8-octane diisocyanate (nonane triisocyanate) or triphenylmethane 4,4′,4″-triisocyanate.

It is additionally possible to use, as the polyisocyanate component, NCO-terminated prepolymers formed from the aforementioned polyisocyanates and polyols.

The molar ratio of NCO groups of the polyisocyanate to NCO-reactive OH groups may, for example, be ≧0.90 to ≦1.15, preferably ≧1.0 to ≦1.1, more preferably ≧1.02 to ≦1.07.

In one embodiment of this process, the reaction of the dispersion with an alkylene oxide in step (b) is performed in the presence of a tertiary amine as a catalyst. Preference is given here to tertiary diamines. Examples thereof are diazabicycloalkanes such as 1,4-diazabicyclo[2.2.2]octane, diazabicycloalkenes such as 1,8-diazabicyclo[5.4.0]undec-7-ene, triamines such as pentamethylenediethylenetriamine, and bis(2-dialkylaminoalkyl)ethers such as bis(2-dimethylaminoethyl)ether. Especially the bis(2-dimethylaminoethyl)ether catalyst leads to the effect that only one molecule of the epoxide reacts with the carboxyl group, such that there is no formation of polyether chains. It has additionally been found that good dispersions are obtained in systems comprising the bis(2-dimethylaminoethyl)ether catalyst.

In a further embodiment of this process, the polyol in step (a) is a polyether polyol and/or a polyester polyol. Preferred polyether polyols have hydroxyl numbers of ≧25 mg KOH/g to ≦550 mg KOH/g, advantageously of ≧100 mg KOH/g to ≦520 mg KOH/g. It is favorable when the polyether polyols are formed exclusively or predominantly on the basis of propylene oxide. Preferred polyester polyols have hydroxyl numbers of ≧100 mg KOH/g to ≦mg KOH/g, advantageously of ≧200 mg KOH/g to ≦500 mg KOH/g. It is possible that the polyols mentioned have molar masses in the range from ≧250 to ≦5000 g/mol, preferably ≧400 to ≦3500 g/mol, and a functionality between ≧1.8 and ≦6, preferably between ≧1.95 and ≦3.5.

In a further embodiment of this process, R1 and R2 in the epoxide in step (b) are each independently hydrogen, methyl, ethyl, n-propyl, isopropyl, n-butyl, sec-butyl, isobutyl, tert-butyl and/or phenyl, or together are —(CH₂)₄—. The epoxide in step (b) is preferably a terminal alkylene oxide. In that case, at least one of the R1 and R2 radicals is hydrogen. Examples of such alkylene oxides are ethylene oxide, propylene oxide and n-butylene oxide. Preference is given here to ethylene oxide and propylene oxide.

In a further embodiment of this process, the polyisocyanate in step (c) is a polyisocyanate based on diphenyl 4,4′-diisocyanate (MDI). The fact that the polyisocyanate is based on MDI means that it is monomeric, polycyclic or polymeric MDI. For example, it may have an NCO content of ≧25% by weight to ≦35% by weight. The NCO content may also be within a range from ≧29% by weight to ≦31% by weight.

The present invention further provides a polyurethane polymer comprising carbon nanotubes, wherein at least some of the carbon nanotubes are bonded covalently to the polyurethane polymer. The polyurethane polymer is notable in that urethane bonds formed from hydroxyalkyl ester groups bonded covalently to the surface of the carbon nanotubes and free isocyanate groups of the polyurethane polymer are present between the carbon nanotubes and the polyurethane polymer, said urethane bonds being selected from the group of the general formulae (3) and/or (4):

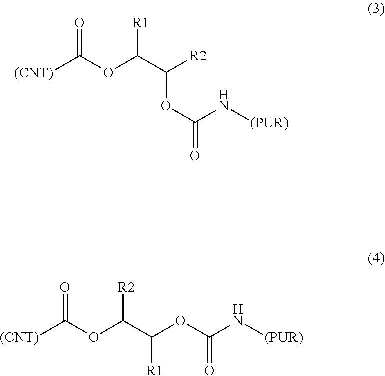

where (CNT) represents the surface of the carbon nanotube, (PUR) represents a polyurethane polymer and R1 and R2 are each independently hydrogen, an alkyl radical or an aryl radical.

The structures of the general formulae (3) and (4) can be derived proceeding from inventive carbon nanotubes comprising hydroxyl groups according to the general formulae (1) and (2), by reaction of the free OH group with an isocyanate group, which achieves attachment to the polyurethane polymer. Consequently, the inventive polyurethane polymer can also be described such that it is obtainable from the above process according to the invention.

In the inventive polyurethane polymer, stiffening can be observed compared to a polyurethane not provided with carbon nanotubes, and also compared to one comprising carbon nanotubes not functionalized in accordance with the invention. Without being bound to a theory, is it assumed that the reactive attachment of the polyurethane to the surface of the carbon nanotube results in a more homogeneous distribution within the polymer matrix and, at the same time, the fixed attachment of the polyurethane favorably influences the mechanical properties.

Details regarding the carbon nanotubes (CNT), the R1 and R2 radicals and the reaction components from which the polyurethane polymer (PUR) is obtained have already been described above, including preferred embodiments. For avoidance of repetition, reference is made completely thereto in connection with the inventive polyurethane polymer.

The proportion of the carbon nanotubes in the inventive polyurethane polymer may, for example, be ≧0.1% by weight to ≦5% by weight, preferably ≧0.5% by weight to ≦1% by weight. Such a low proportion of the carbon nanotubes can already lead to a noticeable reinforcement of the material.

The inventive polyurethane polymer may have, for example, a modulus of elasticity of ≧1 N/mm² to ≦10 000 N/mm². In one embodiment, it is an elastomer with a modulus of elasticity of ≧10 N/mm² to ≦5000 N/mm², preferably of ≧100 N/mm² to ≦1000 N/mm². The modulus of elasticity can be determined as the slope at the operating point of the stress-strain curve from the tensile test according to DIN 53 504 with minimal deformation between 0.025% and 0.05% strain.

The present invention further provides for the use of inventive carbon nanotubes comprising hydroxyl groups for preparation of polymers comprising carbon nanotubes. This may, for example, be a process in which the inventive carbon nanotubes are added to the polymer itself or to the reaction mixture from which the polymer is obtained. Such a reaction mixture can lead to a polyurethane. Another example is a reaction mixture which leads to an epoxy resin. The free hydroxyl groups can be used to bind the inventive carbon nanotubes covalently in the polymer matrix. However, it is likewise possible that the inventive carbon nanotubes are present without covalent attachment in the polymer matrix.

The present invention is illustrated further by the examples which follow. In these examples, the materials and abbreviations used are defined as follows:
Desmophen® VP.PU 22HS51: bifunctional polyether polyol with an OH number of 112 mg KOH/g (Bayer MaterialScience)
Desmodur® CD-S: modified polyisocyanate based on diphenyl 4,4′-diisocyanate, with an NCO content of 29.5% (Bayer MaterialScience)
DABCO 33-LV: amine catalyst; 1,4-diazabicyclo[2.2.2]octane; 33% solution in propylene glycol (Air Products)
Niax A 1: amine catalyst; bis(2-dimethylaminoethyl)ether; 70% solution in dipropylene glycol (Momentive Performance Materials Inc.)
DBTL: dibutyltin dilaurate
DPG: dipropylene glycol
Oxidized CNTs: oxidized carbon nanotubes of the Baytubes® C150P type (Bayer MaterialScience), in which carboxyl groups have been introduced on the surface by known processes

The mechanical properties of the elastomers obtained in the examples were determined by the following standards:
Hardness [Shore A]: DIN 53 505
Tensile strength [MPa]: DIN 53 504
Maximum strain [%]: DIN 53 504
Modulus of elasticity [N/mm²]: as the slope at the operating point of the stress-strain curve from the tensile test to DIN 53 504 with minimal deformation between 0.025% and 0.05% strain

### General Remarks Regarding the Examples:

The Baytubes C150P (multiwall carbon nanotubes) oxidized by the HNO₃ process have a carboxyl group concentration of 0.5 mmol/g (conductometric titration). These oxidized carbon nanotubes were introduced into a carrier polyether polyol in two different concentrations (1% by weight and 3% by weight). For this purpose, they were coarsely predispersed with a rotor/stator system (T 18 basic ULTRA-TURRAX®, IKA Werke GmbH & Co. KG, Staufen, Germany) in order to obtain a first distribution of the nanotubes in the medium and wetting with the medium. Simply stirring in the nanotubes was insufficient for the preparation of a stable dispersion which can be used for a subsequent chemical reaction.

This dispersion was initially charged in an appropriate reactor with an aminic catalyst and admixed with propylene oxide. The course of the reaction was observed by the acid number determination. On completion of reaction, excess propylene oxide was distilled off.

The polyol dispersion with PO-functionalized carbon nanotubes thus obtained, directly before the reaction with the isocyanate, was sheared once again in a rotor/stator system (UltraTURRAX) and treated with ultrasound (HD 3200 sonicator probe, BANDELIN electronic GmbH & Co. KG, Berlin, Germany) in order to ensure maximum quality of dispersion.

This fine dispersion was degassed briefly under reduced pressure both before and after the addition of the catalyst. The isocyanate was stirred briefly with the dispersion. This reaction mixture was poured into a two-piece metal mold and heated at 70° C.

The carbon nanotube-containing polyurethane elastomers thus obtained were analyzed for their hardness and tensile strength.

### EXAMPLE 1

### Predispersion, 1% by Weight, Dispersion 1

A 150 ml beaker was initially charged with 1 g of the oxidized CNTs, and 99 g of Desmophen® VP.PU 22HS51 were added. This mixture was sheared at 24 000 rpm with a rotor/stator system (T 18 basic ULTRA-TURRAX®, IKA Werke GmbH & Co. KG, Staufen, Germany) while cooling with ice-water for 2 minutes.

### EXAMPLE 2

### Predispersion, 3% by Weight, Dispersion 2

A 150 ml beaker was initially charged with 3 g of the oxidized CNTs, and 97 g of Desmophen® VP.PU 22HS51 were added. This mixture was sheared at 24 000 rpm with a rotor/stator system (T 18 basic ULTRA-TURRAX®, IKA Werke GmbH & Co. KG, Staufen, Germany) while cooling with ice-water for 2 minutes.

### EXAMPLE 3

### Functionalization, Dispersion 3

A 500 ml pressurized glass reactor was initially charged with 227 g of a 1% by weight dispersion of the oxidized CNTs in Desmophen® VP.PU 22HS51 (dispersion 1) with 0.227 g of bis(2-dimethylaminoethyl)ether under protective gas (nitrogen), and then heated to 125° C. Subsequently, 10 g of propylene oxide were metered in, in the course of which the reactor pressure rose to 3.0 bar (absolute). After 5 h of reaction while stirring at 125° C., volatile components were distilled off at 90° C. (1 mbar) and the reaction mixture was then cooled to room temperature. At the start of the reaction, the acid number of the reaction mixture was 0.55 mg KOH/g, and at the end of the reaction 0.08 mg KOH/g.

### EXAMPLE 4

### Functionalization, Dispersion 4

A 500 ml pressurized glass reactor was initially charged with 227 g of a 3% by weight dispersion of the oxidized CNTs in Desmophen® VP.PU 22HS51 (dispersion 2) with 0.227 g of bis(2-dimethylaminoethyl)ether under protective gas (nitrogen), and then heated to 125° C. Subsequently, 10 g of propylene oxide were metered in, in the course of which the reactor pressure rose to 3.0 bar (absolute). After 5 h of reaction while stirring at 125° C., volatile components were distilled off at 90° C. (1 mbar) and the reaction mixture was then cooled to room temperature. At the start of the reaction, the acid number of the reaction mixture was 0.86 mg KOH/g, and at the end of the reaction 0.04 mg KOH/g.

### EXAMPLE 5

### Fine Dispersion, 1% by Weight, Dispersion 3A

A 150 ml beaker was initially charged with 90 g of dispersion 3. This mixture was sheared at 24 000 rpm with a rotor/stator system (T 18 basic ULTRA-TURRAX®, IKA Werke GmbH & Co. KG, Staufen, Germany) while cooling with ice-water for 2 minutes, and then treated with ultrasound (HD 3200 sonicator probe, BANDELIN electronic GmbH & Co. KG, Berlin, Germany), likewise while cooling with ice, until a total energy input of 75 kJ. The dispersion 3A thus obtained was used directly for the further reaction with an isocyanate.

### EXAMPLE 6

### Fine Dispersion, 3% by Weight, Dispersion 4A

A 150 ml beaker was initially charged with 90 g of dispersion 4. This mixture was sheared at 24 000 rpm with a rotor/stator system (T 18 basic ULTRA-TURRAX®, IKA Werke GmbH & Co. KG, Staufen, Germany) while cooling with ice-water for 2 minutes, and then treated with ultrasound (HD 3200 sonicator probe, BANDELIN electronic GmbH & Co. KG, Berlin, Germany), likewise while cooling with ice, until a total energy input of 75 kJ. The dispersion 4A thus obtained was used directly for the further reaction with an isocyanate.

### EXAMPLE 7

### Preparation of the PUR Elastomers

The polyols (with and without Baytubes C150P) or the polyol dispersions comprising oxidized CNTs (3A or 4A) were initially charged in a 1 l flanged pot, and degassed briefly both before and after the addition of catalyst. The isocyanate was stirred briefly at room temperature, and the reaction mixture was poured out into a two-part metal mold and then subjected to a heating cycle.

The mixtures PUR1 to PUR6 are reproduced individually in the table below.

| Substance | PUR1 | PUR2 | PUR3 | PUR4 | PUR5 | PUR6 |
|---|---|---|---|---|---|---|
| Desmophen ® | 99.26 | 99.26 | — | 96.30 | 96.30 | — |
| VP.PU 22HS51 | | | | | | |
| Dispersion 3A | — | — | 100.26 | — | — | — |
| Dispersion 4A | — | — | — | — | — | 99.30 |
| DPG | 0.14 | 0.14 | — | 0.45 | 0.45 | — |
| DBTL | 0.02 | 0.02 | 0.02 | 0.02 | 0.02 | 0.02 |
| Dabco 33-LV | 0.50 | 0.50 | 0.50 | 0.50 | 0.50 | 0.50 |
| Niax A1 | 0.10 | 0.10 | — | 0.10 | 0.10 | — |
| Desmodur ® | 30.73 | 30.73 | 30.66 | 30.55 | 30.55 | 30.37 |
| CD-S | | | | | | |
| Baytubes ® | — | 1.00 | — | — | 2.98 | — |
| C150P | | | | | | |
| Index | 105.0 | 105.0 | 105.0 | 105.0 | 105.0 | 105.0 |

The mechanical properties of the resulting elastomers PUR1 to PURE are listed in the two tables below.

| | PUR1 | PUR2 | PUR3 |
|---|---|---|---|
| Nanotube type | — | Baytubes ® C150P | Inventive |
| c(Nanotube) | — | 0.76 | 0.76 |
| [% by weight] | | | |
| Hardness | 51 | 51 | 56 |
| [Shore A] | | | |
| Tensile | 2.0 ± 0.3 | 2.0 ± 0.4 | 2.4 ± 0.3 |
| strength | | | |
| [MPa] | | | |
| Maximum | 334.7 ± 42.3 | 347.8 ± 56.7 | 312.1 ± 30.9 |
| strain [%] | | | |
| Modulus of | 1.9 ± 0.1 | 1.8 ± 0.1 | 2.0 ± 0.1 |
| elasticity | | | |
| [N/mm²] | | | |
| | PUR4 | PUR5 | PUR6 |
| Nanotube type | — | Baytubes ® C150P | Inventive |
| c(Nanotube) | — | 2.28 | 2.28 |
| [% by weight] | | | |
| Hardness | 54 | 60 | 64 |
| [Shore A] | | | |
| Tensile | 2.1 ± 0.2 | 2.6 ± 0.1 | 3.9 ± 0.4 |
| strength | | | |
| [MPa] | | | |
| Maximum | 339.0 ± 28.6 | 243.0 ± 1.7 | 227.3 ± 24.4 |
| strain [%] | | | |
| Modulus of | 1.9 ± 0.0 | 2.8 ± 0.2 | 3.7 ± 0.1 |
| elasticity | | | |
| [N/mm²] | | | |

For better comparability, the table which follows shows the percentage change in the material characteristics determined for the elastomers filled with carbon nanotubes compared to the corresponding unfilled elastomer PUR1 or PUR4.

| | PUR2 | PUR3 | PUR5 | PUR6 |
|---|---|---|---|---|
| Hardness | 0% | 10% | 11% | 19% |
| Tensile strength | 1.1% | 19.5% | 21.7% | 82.9% |
| Maximum strain | 3.9% | −6.7% | −28.3% | −33.0% |
| Modulus of elasticity | −3.1% | 5.9% | 48.1% | 93.2% |

The reinforcing influence of the carbon nanotubes functionalized in accordance with the invention on the polyurethane elastomer is evident here. Compared to the unfilled elastomer PUR1, the elastomer PUR3 modified with inventive carbon nanotubes had a Shore A hardness greater by 10%, a tensile strength higher by 19.5%, a maximum strain reduced by 6.7% and a modulus of elasticity higher by 5.9%. The elastomer PUR2 comprising the carbon nanotubes of the Baytubes®C150P type which had not been functionalized in any way had merely no increase in the Shore hardness compared to the unfilled elastomer PUR1, a marginal increase in tensile strength, actually an increase in the maximum strain, and a lowering of the modulus of elasticity.

In the same way, the elastomer PUR6 modified with the inventive carbon nanotubes, compared to the corresponding unfilled elastomer PUR4, had a Shore A hardness greater by 19%, a tensile strength higher by 82.9%, a maximum strain lower by 33% and a modulus of elasticity higher by 93.2%. The elastomer PUR5 comprising the carbon nanotubes of the Baytubes® C150P type which had not been functionalized in any way had only a slight increase in the Shore hardness and in tensile strength and a small lowering of the maximum strain compared to the unfilled elastomer PUR4, and a much smaller increase in the modulus of elasticity compared to the inventive elastomer PUR6 modified with carbon nanotubes.

1.-15. (canceled)

16. A carbon nanotube comprising hydroxyl groups, wherein an hydroxyalkyl ester group is covalently bonded to a surface of the carbon nanotube, and wherein the hydroxyalkyl ester group is selected from the group consisting of formula (1) and formula (2): 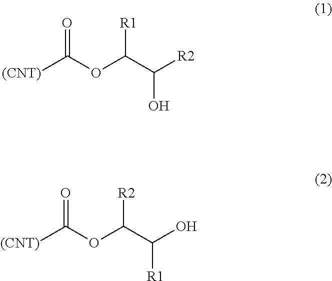
wherein CNT represents the surface of the carbon nanotube; and
R1 and R2 represent, independently of one another, hydrogen, an alkyl radical, or an aryl radical.

17. The carbon nanotube comprising hydroxyl groups as claimed in claim 16, wherein R1 and R2 represent, independently of one another, hydrogen, methyl, ethyl, n-propyl, isopropyl, n-butyl, sec-butyl, isobutyl, tert-butyl and/or phenyl, or together are —(CH₂)₄—.

18. The carbon nanotube comprising hydroxyl groups as claimed in claim 16, wherein the carbon nanotube is a single-wall carbon nanotube, a multiwall carbon nanotube, or a multiwall carbon nanotube of the cylinder type, of the scroll type, of the multiscroll type and/or with onion-like structure.

19. A process for preparing the carbon nanotube comprising hydroxyl groups as claimed in claim 16, comprising the steps of:
(a) providing a carbon nanotube comprising carboxyl groups covalently bonded to a surface of the carbon nanotube; and
(b) reacting the carbon nanotube from step (a) with an epoxide of the formula 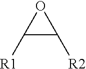
wherein R1 and R2 represent, independently of one another, hydrogen, an alkyl radical or an aryl radical.

20. The process as claimed in claim 19, wherein the reaction of the carbon nanotube in step (b) is performed in the presence of a tertiary amine as a catalyst.

21. The process as claimed in claim 19, wherein R1 and R2 in the epoxide in step b) represent, independently of one another, hydrogen, methyl, ethyl, n-propyl, isopropyl, n-butyl, sec-butyl, isobutyl, tert-butyl and/or phenyl, or together are —(CH₂)₄—.

22. The process as claimed in claim 19, wherein the carbon nanotube in step (a) is a single-wall carbon nanotube, a multiwall carbon nanotube, or a multiwall carbon nanotube of the cylinder type, of the scroll type, of the multiscroll type and/or with onion-like structure.

23. The process as claimed in claim 19, wherein the reaction in step (b) is performed at a temperature of from 100° C. to 150° C.

24. A process for preparing polyurethane polymers comprising carbon nanotubes, comprising the steps of:
(a) providing a dispersion of carbon nanotubes in a polyol, wherein the carbon nanotubes have carboxyl groups covalently bonded to a surface of the carbon nanotubes;
(b) reacting the dispersion from step (a) with an epoxide of the formula: 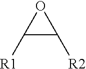
wherein R1 and R2 represent, independently of one another, hydrogen, an alkyl radical or an aryl radical; and
(c) reacting the dispersion obtained in step (b) with a polyisocyanate.

25. The process as claimed in claim 24, wherein the reaction of the dispersion with an alkylene oxide in step (b) is performed in the presence of a tertiary amine as a catalyst.

26. The process as claimed in claim 24, wherein the polyol in step (a) comprises a polyether polyol and/or a polyester polyol.

27. The process as claimed in claim 24, wherein R1 and R2 in the epoxide in step (b) represent, independently of one another, hydrogen, methyl, ethyl, n-propyl, isopropyl, n-butyl, sec-butyl, isobutyl, tert-butyl and/or phenyl, or together are —(CH₂)₄—.

28. The process as claimed in claim 24, wherein the polyisocyanate in step (c) comprises a polyisocyanate based on diphenyl 4,4′-diisocyanate.

29. A polyurethane polymer comprising carbon nanotubes, wherein at least some of the carbon nanotubes are bonded covalently to the polyurethane polymer, wherein the bond between the carbon nanotubes and the polyurethane polymer are formed from hydroxyalkyl ester groups bonded covalently to the surface of the carbon nanotubes and free isocyanate groups of the polyurethane polymer, wherein said bond between the carbon nanotubes and the polyurethane polymer is selected from the group consisting of the formula (3), (4), and mixtures thereof: 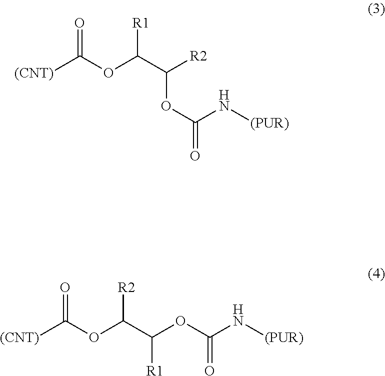
wherein (CNT) represents the surface of the carbon nanotube;
(PUR) represents a polyurethane polymer; and
R1 and R2 represent, independently of one another, hydrogen, an alkyl radical or an aryl radical.
